(19) United States
(12) Patent Application Publication
(10) Pub. No.: US 2008/0147040 A1
(43) Pub. Date: Jun. 19, 2008

(54) Catheters Having Linear Electrode Arrays and Their Methods of Use

(75) Inventor: Maria Dikshteyn, Mayfield Heights, OH (US)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc. A Delaware Corporation, Santa Rosa, CA (US)

(21) Appl. No.: 11/610,092

(22) Filed: Dec. 13, 2006

(51) Int. Cl. A61M 25/01; A61B 5/00

(52) U.S. Cl. 604/506; 600/300

(57) ABSTRACT

Catheter devices, systems and methods wherein linear electrode arrays are disposed on catheters that penetrate through tissue such that the electrodes of the array may be used to sense properties of surrounding tissue or body fluid. The invention is useable for various purposes, including the delivery of a substance, article or device to a specific target location within the body of a human or animal subject.

### FIELD OF THE INVENTION

The present invention relates generally to methods and apparatus for medical treatment and more particularly to catheter devices, systems and methods wherein linear electrode arrays are disposed on catheters that penetrate through tissue such that the electrodes of the array may be used to sense properties of surrounding tissue or body fluid.

### BACKGROUND

Targeted Substance Delivery

In a variety of situations it is desirable to deliver therapeutic or diagnostic substances (e.g., drugs, biologics, cells, genes, fillers, tissue adhesives, etc.), articles (e.g., implants, beads, coils, pellets, etc.) or devices (e.g., guidewires, sensors, etc.) to specific locations within body of a human or animal subject. Examples of target locations to which substances, articles and/or devices may be delivered include: organs, body lumens, myocardial tissue, infarcted or necrotic tissue, brain tissue skeletal muscle, nerves, blood vessel walls, tumors and other normal or pathological tissues. Also, in some instances, it may be desirable to advance a catheter into or adjacent to a previously implanted device (e.g., a refillable drug delivery reservoir, a prosthetic device, a fluid filled implant, etc.)to deliver a substance (e.g., a refill quantity of a drug or fluid, a lubricant, a filler material, etc.), article (e.g., a small battery or other item) or some ancillary apparatus (e.g., a power supply wire, etc.) to that previously implanted device.

Some catheters and implantable substance delivery devices (e.g., drug eluting stents) have been used to indirectly deliver drugs or substances to specific target locations within the body by releasing the drug within the lumen of a nearby blood vessel and allowing the drug to diffuse through the blood vessel wall or distribute through downstream capillaries, to the desired target location.

The prior art has also included catheter devices that may be used for delivering substances, articles or devices directly into interstitial target locations by guided advancement of a penetrating catheter into the lumen of a blood vessel and subsequently advancing a penetrator such as a hollow needle from the catheter, into or through the wall of the blood vessel in which the catheter is positioned and through any intervening tissue to the target site. The desired substance, article or device may then be delivered.

Particular interest has developed in methods for controlled or targeted delivery of substances such as drugs (e.g., chemotherapeutic agents), gene therapy compositions (e.g., plasmids, viral vectors genetically modified cells, naked DNA), biological factors (e.g., angiogenic factors, nerve growth factors, other cell growth factors other proteins), monoclonal antibodies, or specific cell types (e.g., stem cells or other progenator cells, pancreatic islet cells, dopamine secreting neurons, endothelial cells, myocardial cells, other myocytes, etc) into interstitial target locations for the purpose of treating diseases such as myocardial ischemia, solid tumor types of cancer, parkansonism, diabetes, etc.

Specifically, in the treatment of myocardial ischemia, research has indicated that introduction of certain angiogenic substances into ischemic areas of myocardium may result in therapeutic angiogenesis in patients who suffer from clinically significant coronary artery disease. Generally speaking, the term “angiogenesis” refers to the creation of new capillaries and/or blood vessels within the parenchyma of an organ, within a tumor or within an area of tissue (e.g., myocardium). Angiogenesis is believed to occur as a multi-step process in which endothelial cells focally degrade and invade through their own basement membrane, migrate through interstitial stroma toward an angiogenic stimulus, proliferate proximal to the migrating tip, organize into blood vessels, sand reattach to newly synthesized basement membrane. The term “therapeutic angiogenesis” generally refers to the administration of angiogenic substances or treatments to promote creation of new blood vessels or capillaries in tissues that previously lacked sufficient blood flow.

Various approaches have heretofore been used for delivery of angiogenic substances into the myocardium. One approach is the use a tissue penetrating device, such as a laser, to create penetration tracts or transmyocardial (TMR) channels which extend from either the epicardial (outer) surface or endocardial (inner)surface of the heart into the myocardium, and to then inject quantities of angiogenic substances into those TMR channels. Examples of this approach are described in U.S. Pat. Nos. 5,925,012 (Murphy-Chutorian, et al.), 5,999,678 (Murphy-Chutorian, et al.) and 6,106,520 (Laufer, et al.).

Catheters Having Electrodes for Sensing Properties of Tissue

Various electrophysiological diagnostic catheters have been known in the prior art. A typical electrophysiological diagnostic catheter comprises a flexible catheter that may be advanced through coronary blood vessels and/or chambers of the heart while one or more electrodes on the catheter are used to sense electrophysiological signals in tissue surrounding the particular coronary blood vessel in which the catheter is positioned. The electrophysiological signals sensed by the catheter-mounted electrode(s) are then used to map the electrophysiological activity in regions of the myocardium of interest and to diagnose arrhythmogenic foci or lesions in conduction pathways that may be repaired by electrical ablation therapy. Examples of commercially available electrophysiological catheters useable for mapping and diagnosis include but are not limited to the TORQR® series fixed curve diagnostic catheters, MARINR® series coronary sinus diagnostic catheters, SOLOIST™ series fixed curve diagnostic catheters, and STABLEMAPR™ diagnostic catheters (Medtronic Corporation, Minneapolis, Minn.) and the Biosense-Webster fixed curve catheters, CRISTA CATH deflectable diagnostic catheters, HALO XP 20 pole deflectable mapping catheters and LASSO circular mapping catheters (Johnson & Johnson, New Brunswick, N.J.).

The electrophysiological diagnostic and mapping catheters of the prior art have not typically been used to penetrate through tissues, but rather are designed to move within blood vessel lumens and chambers of the heat without penetrating into surrounding tissues.

There remains a need in the art for the development of new catheters that are capable of penetrating or advancing through tissue, are capable of providing information about the surrounding environment for the purpose of delivering substances, articles or devices to specific interstitial target locations.

### SUMMARY OF THE INVENTION

In accordance with the present invention there is provided a catheter that incorporates a linear electrode array that is useable to sense properties of tissue or body fluid into which the catheter is advanced. In general, such catheter device comprises i) an elongate catheter body (rigid or flexible) having a lumen and a distal end that penetrates through tissue, ii) a linear electrode array (e.g., a plurality of electrodes arranged in a row at spaced apart locations) on or in the catheter body, such electrode array being operative to sense a property of tissue or body fluid and to generate signals in response to the sensed property and iii) a display apparatus for displaying indicia of the sensed property. The property of tissue or body fluid sensed by the electrodes may be any desired electrical, chemical, thermal, physiological or other property. In some embodiments, the electrodes will sense electrophysiological signals within tissue, thereby distinguishing tissues of different types and/or distinguishing between healthy tissue (e.g., normal myocardial, brain or other tissue) and diseased tissue (e.g., ischemic, necrotic or infarcted areas of the myocardium, brain or other tissue).

Further in accordance with the invention, there is provided a system for delivering a substance, article or device to a desired target location within the body of a human or animal subject. In general, such system comprises a linear electrode array equipped catheter of the type summarized in the immediately preceding paragraph in combination with another tissue penetrating catheter device. The other tissue penetrating catheter device is positionable within a body lumen (e.g., a blood vessel, urethra, lymphatic or other natural or man made luminal structure within the body) and a tissue penetrating member (e.g., a hollow needle or wire having a sharp tip) is advancable from the catheter to a first location outside of the body lumen in which the catheter is positioned. The linear electrode array equipped catheter device is then advanceable through or over the tissue penetrating member such that the linear electrode array equipped catheter device will advance into tissue or body fluid and the electrodes of the linear electrode array will sense a property of that tissue or body fluid.

In some embodiments, as described herein, the other tissue penetrating catheter may incorporate orientation apparatus (e.g., imageable markers, sensors, on board imaging apparatus, etc.) for determining the rotational orientation of the catheter and/or the expected trajectory or path on which the tissue penetrating member will advance such that the operator may adjust the position and/or rotational orientation of the other tissue penetrating catheter within the body lumen to substantially ensure that subsequent advancement of the tissue penetrating member will cause the tissue penetrating member to advance to the intended first location (e.g., in the direction of the target location) and not some other location.

Examples of tissue penetrating catheters that incorporate such orientation apparatus include but are not limited to those described in U.S. Pat. Nos. 5,830,222 (Makower), 6,068,638 (Makower), 6,159,225 (Makower), 6,190,353 (Makower, et al.), 6,283,951 (Flaherty, et al.), 6,375,615 (Flaherty, et al.), 6,508,824 (Flaherty, et al.), 6,544,230 (Flaherty, et al.), 6,579,311 (Makower), 6,602,241 (Makower, et al.), 6,655,386 (Makower, et al.), 6,660,024 (Flaherty, et al.), 6,685,648 (Flaherty, et al.), 6,709,444 (Makower), 6,726,677 (Flaherty, et al.) and 6,746,464 (Makower), the entire disclosure of each such United States patent being expressly incorporated herein by reference.

Still further in accordance with the invention, there is provided a method for delivering a substance, article or device to a target location within the body of a human or animal subject. This method generally comprises the steps of i) providing a linear electrode array equipped catheter device as summarized above; ii) inserting the linear electrode array equipped catheter device into the subject's body and advancing it through tissue or body fluid such that the electrodes of the linear electrode array will sense a property of the tissue or body fluid and the display apparatus will display indicia of the property sensed by each electrode, iii) determining on the basis of the indicia displayed by the display device when the catheter body is positioned such that introduction of the substance, article or device through the lumen of the catheter body will result in delivery of the substance, article or device to the target location and iv) delivering the substance, article or device through the lumen of the catheter body to the target location.

In some applications of this method, another tissue penetrating catheter device may be initially positioned within a body lumen and a penetrator may be advanced from that intraluminal catheter to a fist location outside of the body lumen in which that catheter is positioned. Thereafter, the linear electrode array equipped catheter is advanced through or over that tissue penetrator, through intervening tissue, and to the intended target location using information provided by the linear electrode array to determine when the linear electrode array equipped catheter is properly positioned for delivery of the intended substance, article or device.

Further aspects, details and embodiments of the present invention will be understood by those of skill in the art upon reading the following detailed description of the invention and the accompanying drawings.

### BRIEF DESCRIPTION OF THE DRAWINGS

FIG. 1 is a perspective view of one embodiment of a linear electrode array-equipped delivery catheter of the present invention.

FIG. 1A is side view of a system of the present invention comprising the linear electrode array-equipped delivery catheter of FIG. 1 in combination with a transluminal tissue penetrating catheter having a penetrator through which the linear electrode array-equipped delivery catheter is advanced.

FIG. 2 is an enlarged view of Region 2 of FIG. 1A.

FIG. 3 is an enlarged schematic diagram of one example of a signal processing and display apparatus to which the linear electrode array-equipped delivery catheter of the present invention may be attached.

FIG. 4 shows a diagram of a human subject in whom the catheter system of FIG. 2 has been inserted and positioned to perform a procedure wherein a therapeutic or diagnostic substance is delivered to a target location within the myocardium of the subject's heart.

FIG. 4A is an enlarged view of the heart of the human subject shown in FIG. 4.

FIGS. 4B-4C are transmural sectional views through region 4B of FIG. 4A showing steps in a method for delivering an substance, article or device to a target location within the myocardium of the subject's heart using the system of FIG. 1A.

FIGS. 4D-4E are transmural sectional views through region 4B of FIG. 4A showing steps in a method whereby the linear electrode array equipped catheter of the present invention may be used to distinguish between different regions of the heart (e.g., myocardium, endocardium, ventricle).

### DETAILED DESCRIPTION

The following detailed description, the accompanying drawings are intended to describe some, but not necessarily all, examples or embodiments of the invention. The contents of this detailed description and accompanying drawings do not limit the scope of the invention in any way.

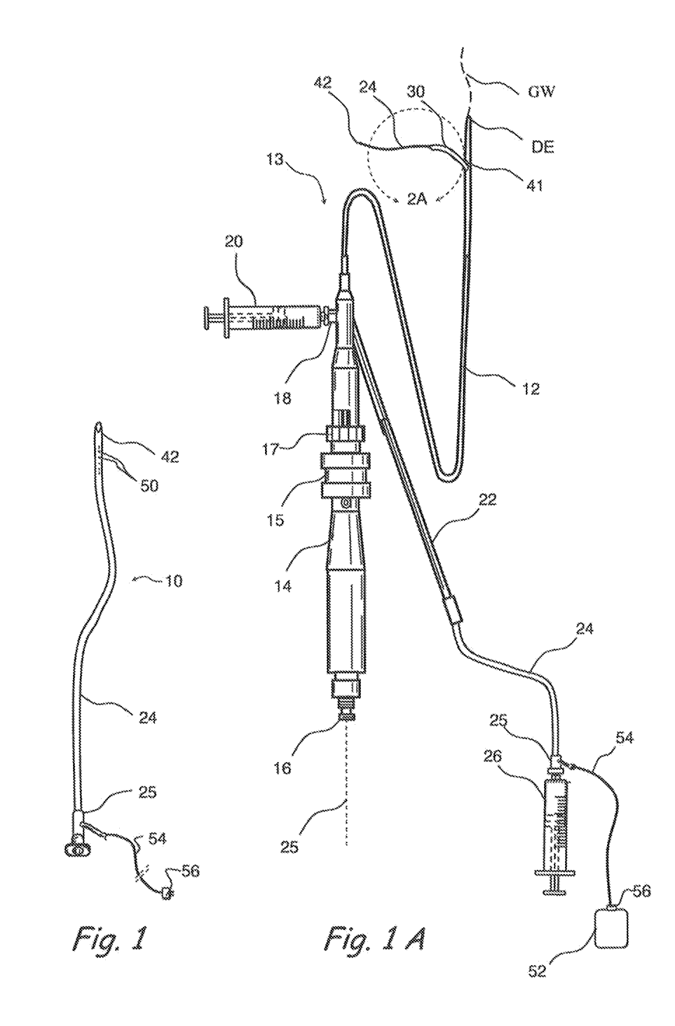

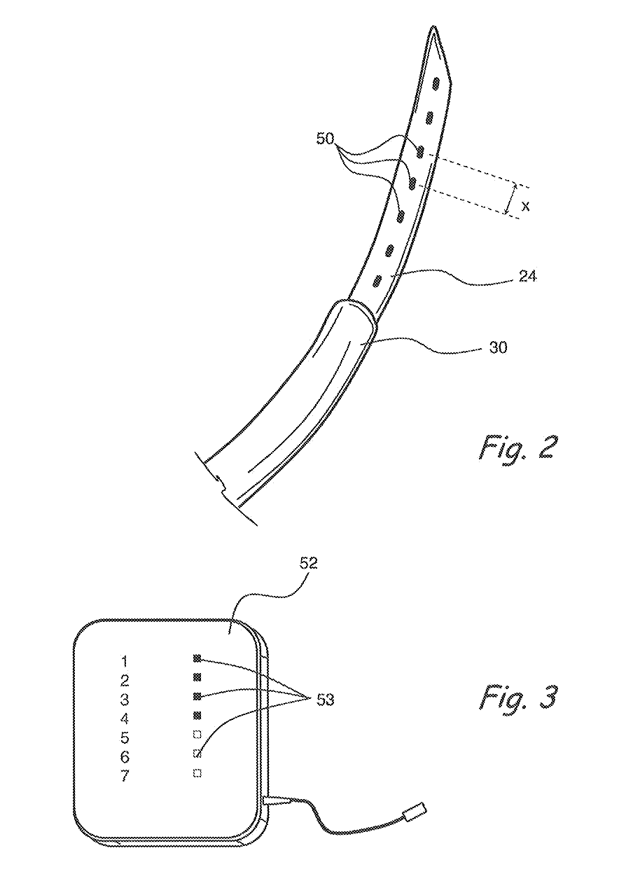

FIGS. 1 and 2 show a linear electrode array equipped catheter device 10 and system 13 of the present invention. The linear electrode array equipped catheter device 10 comprises an elongated, flexible catheter body 24 having a lumen that terminates distally at an open distal end 42. A linear array of electrodes 50 is mounted on or in the catheter body 24, generally parallel to the longitudinal axis of the catheter body 24. In this embodiment the linear array of electrodes 50 comprises seen electrodes 50. The distal-most electrode (i.e., Electrode #1) is located about 2 mm from the distal end 42 of the catheter body 24 and the distal end 42 of the catheter body 24. A proximal hub member 25 is attached to the proximal end of the catheter body 24. A port/Luer connector is formed on the proximal end of hub 25 in communication with the catheter lumen such that a syringe 26 or other apparatus may be attached to the port/Luer connector 55 and used to deliver a substance, article or device in a distal direction through the lumen of the catheter body 24 and out of the open distal end 42 or, alternatively to facilitate aspiration or withdrawal in the proximal direction of a substance, article or device.

A connector cable 54 also extends from the proximal hub 25 and terminates in a connector 56, such as a plug, to connect the linear array of electrodes 50 to a power source/display device 52. In some embodiments, the electrodes 50 may be operative to sense electrophysiological signals in tissue and the power source/display device 52 may provide current to the electrodes 50 and may receive, process and display indicia of the electrophysiological signals sensed by each electrode. For example, as seen in FIG. 3, the power source/display device 52 may provide a separate display indicator for each electrode in the array to display qualitative or quantitative indicia of electrophysiological signals being sensed by each electrode at any given point in time. For example, in the example of FIG. 3, a separate indicator light 53 is provided for each of the seven electrodes. Each indicator light 53 emits light when some predetermined type, frequency or intensity of electrophysiological signal is being sensed by that electrode.

In some embodiments, the individual indicator lights 53 may emit differing colors or intensity of light to indicate quantitative variations in the strength, frequency, wave form or some other parameter of the electrophysiological signal being received by that electrode. In the example shown in FIG. 3, the indicator lights 53 associated with electrodes 1, 2, 3 and 4 are illuminated, thereby indicating that electrophysiological signals meeting some predetermined criteria are being sensed in tissue adjacent to those electrodes while the indicator lights 53 associated with more distally positioned electrodes 5, 6 and 7 are not illuminated thereby indicating that no electrophysiological signals meeting predetermined criteria are being sensed by those electrodes. If the predetermined criteria were defined so as to select for electrophysiological signals associated with non-infarcted myocardium, this would indicate that electrodes 1, 2, 3 and 4 are currently positioned within non-infarcted myocardial tissue while the more distally located electrodes 5, 6 and 7 are positioned within infarcted myocardial tissue. Thus, if a therapeutic substance (e.g., an angiogenic agent, myocytes, myoblasts, etc.) were to be injected through the catheter 10 while it is in its current position, the substance would be delivered into the infarcted myocardium.

In other embodiments, the indicator lights 52 may be programmed to indicate the types of tissue or body fluid that is in contact with each electrode 50 at the present time. In this regard, for example, each indicator light may emit red light when the electrode 50 associated with that indicator light 53 is within myocardial tissue, green light when the electrode 50 associated with that indicator light 53 is within endocardial tissue and blue light when the electrode 50 associated with that indicator light 53 has advanced into a chamber of the heart so as to be surrounded by blood. Moreover, it is to be appreciated that indicator lights 53 as shown in FIG. 3 are just one of many possible indicator types that may be incorporated into the display device 52. For example, in some embodiments, the display 52 may provide a plurality of screens (or a single divided screen) that shows actual electrophysiological waveforms as received by each electrode 50. Alternatively, as a further example, the display 52 may provide a series of bar indicators to indicate the relative intensity of the electrical signal being sensed by each electrode 50.

As shown in FIG. 1A, the linear electrode array equipped catheter device 10 may optionally be used in combination with a transluminal tissue penetrating catheter 13 which comprises an elongated catheter body 12 having a distal end DE, laterally deployable tissue penetrator 30 that advances laterally out of side port 41 formed in catheter body 12. This tissue penetrator 30 may comprise a hollow needle having a lumen through which the linear electrode array equipped catheter 30 is advanceable. This tissue penetrator 30 may be formed of any suitable material, such as elastic or superelastic material (e.g., nickel-titanium allow) and may be biased to a curved configuration, as shown.

A handpiece 14 is provided on the proximal end of the catheter body 12, as shown in FIG. 1. The tissue penetrator 30 is moveable between a retracted position where it is substantially retracted within the catheter body 12 and an extended position wherein it has been longitudinally advanced out of side port 41 such that it extends on a trajectory or path away from the catheter body 12. The handpiece 14 comprises an advancement/retraction knob 15 which may be pushed in the distal direction to advance the penetrator 30 from its retracted position to its extended position and pulled in the proximal direction to retract the penetrator 30 from its extended position to its retracted position. An adjustable stop member 17 limits the extent of distal advancement of the advancement/retraction knob 15, thereby controlling the length from the side port 32 to the distal tip of the penetrator 30 when the penetrator 30 is fully extended.

In the particular embodiment shown in the drawings, a proximal side arm 22 is connected to the proximal end of the lumen of the tissue penetrator 30 such that the catheter body 24 of the linear electrode array equipped catheter device 10 may be inserted therethrough and advanced out of the open distal end of the penetrator 30 as seen in FIG. 1A. In some applications, an optional guidewire GW may be inserted through the lumen of the penetrator 30 and the body 24 of the linear electrode array equipped catheter device 10 may then be advanced over such guidewire GW.

In the depicted embodiment of the intraluminal penetrating catheter 13 has an opening at its distal end and a through lumen that extends from a port 16 on the handpiece 14, through the catheter body 12 and through such open distal end of the catheter body 12. A guidewire GW may pass through this lumen for over-the-wire advancement of this catheter device 13. It will be appreciated that, in some alternative embodiments, the lumen may terminate proximally in a side opening in the catheter body 12, thereby providing a rapid exchange type guidewire lumen. Also in the embodiment shown, an infusion port 18 is optionally formed on the handpiece 14 in communication with the through lumen such that an infusion apparatus 20 (e.g., a syringe, intravenous tube, pump, injector, etc.) may be used to infuse fluid (e.g., saline solution, radiographic contrast medium, etc.) may be used to infuse fluid (e.g., saline solution, radiographic contrast medium, etc.) through lumen and out of the open distal end of the tip member 46. A valve (e.g., a Tuohi-Borst valve) may be provided on proximal port 16 to secure a guidewire GW when desired and/or to form a fluid tight seat at proximal port 16 when fluid is being infused through infusion port 18.

Typically, the penetrator 30 will be advanced to a first location. Such first location will typically be between the body lumen in which the penetrating catheter 13 is positioned and the intended target location to which the desired substance, article or device is to be delivered. As explained in the summary of the invention provided above, some embodiments of eh intraluminal tissue penetrating catheter 13 may incorporate orientation apparatus (e.g., imageable markers, sensors, on board imaging apparatus, etc.) for determining the rotational orientation of the catheter and/or the expected trajectory or path on which the tissue penetrating member will advance such that the operator may adjust the position and/or rotational orientation of the other tissue penetrating catheter within the body lumen to substantially ensure that subsequent advancement of the tissue penetrating member will cause the tissue penetrating member to advance to the intended first location (e.g., in the direction of the target location) and not some other location. In such embodiments, the orientation apparatus may be used to position and rotationally orient that catheter body 12 within the body lumen so that subsequent advancement of the penetrator 30 will cause the penetrator to move in the direction of the intended target location instead of some other unintended direction.

After the penetrator 30 has been advanced to the first location, the catheter body 24 of the linear electrode array equipped catheter device 10 is advanced distal end first through the lumen of the penetrator 30 and further through tissue lying beyond the distal end of the penetrator 30, until it has reached the target location. The distal end 42 of the catheter body 24 is capable of penetrating through tissue. In some embodiments, the distal end 42 of the catheter body 24 may comprise a discrete tissue penetrating distal tip member such as those described in copending United States Published Patent Application No. 200/0173440 (Ser. No. 11/279,771) entitled Microcatheter Devices And Methods For Targeted Substance Delivery filed on Apr. 14, 2006 and U.S. Pat. No. 6,602,241, the entire disclosures of which are expressly incorporated herein by reference. In some embodiments, outlet opening(s) may be formed in the side wall of the catheter body 24 instead of or in addition to the opening formed in the distal end 42.

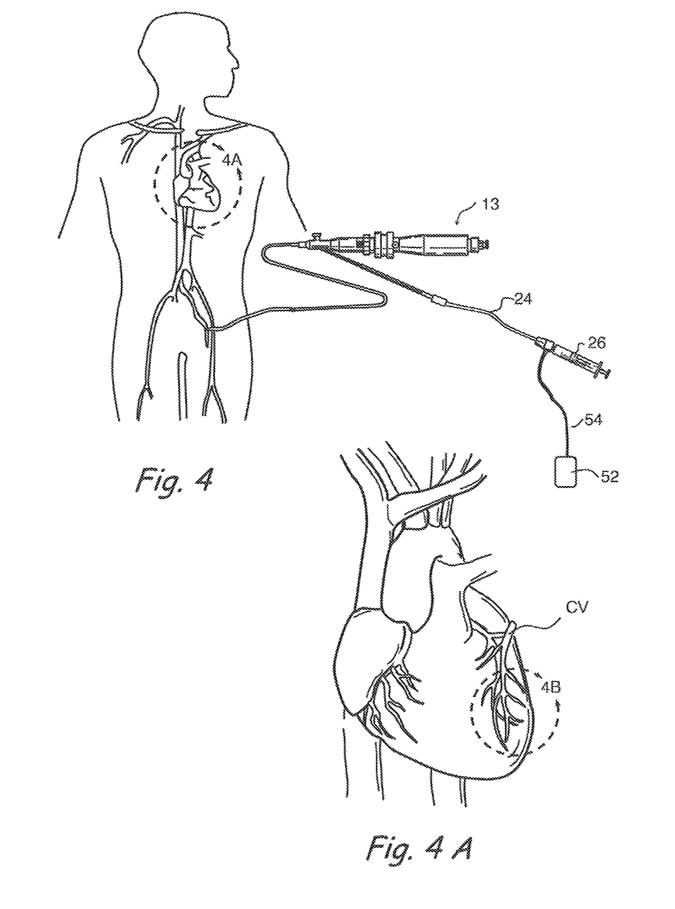

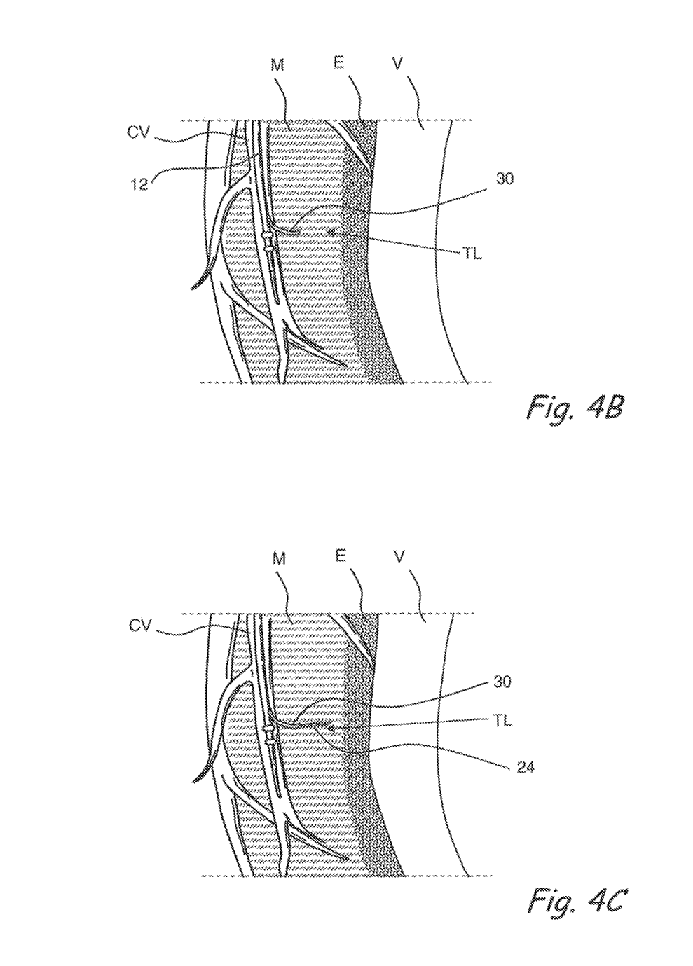

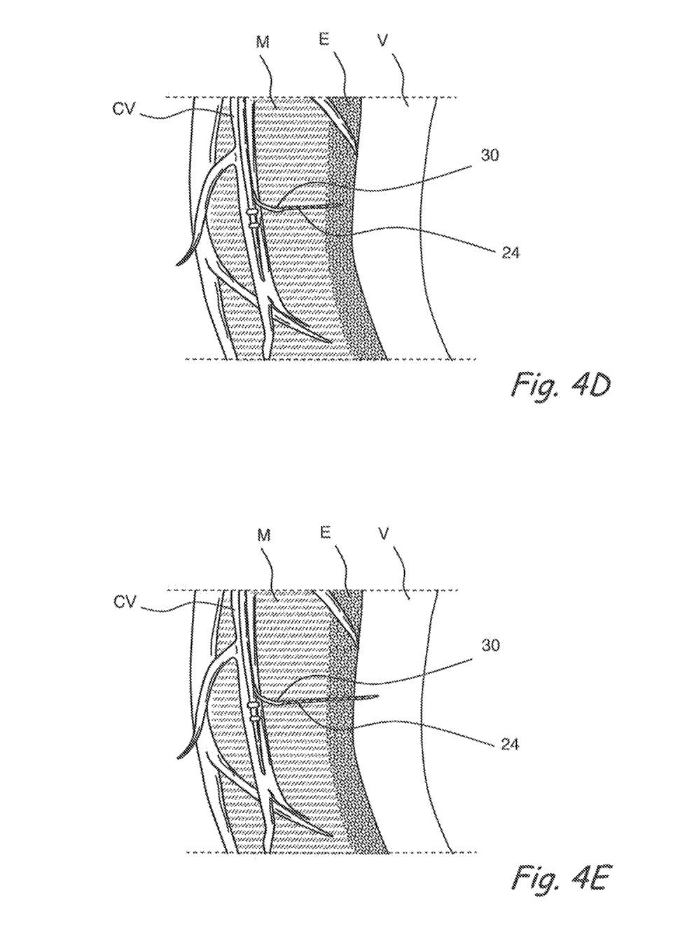

FIGS. 4-4C show an example of a procedure in which the system shown in FIG. 1A is used to deliver a substance, article or device to a target location within the myocardium of the heart of a human subject. As shown, the catheter body 12 of the transluminal tissue penetrating catheter 13 is percutaneously introduced into a femoral blood vessel and advanced, transluminally, through the vasculature to a position within a coronary blood vessel CV located near the intended target location TL. Thereafter, as seen in FIG. 4B, after the penetrating catheter body 12 has been properly positioned and rotationally oriented, the penetrator 30 is advanced through the wall of the coronary blood vessel CV and through a portion of the myocardium M to a first location that is between the coronary blood vessel CV and the target location TL.

As seen in FIG. 4C, the linear electrode array equipped catheter 10 is advanced out of the distal end of the penetrator 30, through myocardial tissue and into the target location. As this catheter 10 is advanced, the electrodes 50 will sense electrophysiological signals and the operator may use the sensed signals as indicated on the display device 52 to determine when the distal end 42 of the catheter body 24 has entered the target location. For example, if the target location is a myocardial infarct, the operator may slowly advance the catheter body 24 until the distal most electrode(s) 50 (i.e., electrode #1 or possibly a plurality of the distal-most electrodes) sense(s) little or no electrophysiological signals, thereby indicating that the distal end 42 of the catheter body 24 has entered the necrotic zone within the infarct. The remaining electrodes will indicate electrophysiological activity consistent with healthy myocardium located between the coronary vessel CV and the infarcted target location TL. A desired substance (e.g., myoblasts, myocytes, angiogenic agents, etc.) may then be delivered through the lumen of catheter 10 directly in to the infarct.

FIGS. 4D and 4E illustrate the manner in which the electrodes 50 may be used to distinguish between tissue types as may be desired when attempting to position the catheter body 24 at a specific location, measure myocardial wall thickness or for other purposes. In the example of FIG. 4D, the catheter body 24 has been advanced through the myocardium M into the endocardium E. As a result, the distal most electrode(s) 50 (i.e., electrode #1 or possibly a plurality of the distal most-electrodes) will sense electrophysiological signals of endocardial tissue while the remaining electrodes 50 will sense electrophysiological signals of myocardial tissue.

In the example of FIG. 4E, when the catheter body 24 is further advanced such that its distal end 42 is within the ventricle V of the heart, the distal most electrode(s) 50 (i.e., electrode #1 or possibly a plurality of the distal most-electrodes) will sense electrophysiological signals indicative of blood within the ventricular chamber, the electrodes located midway along the catheter body 50 (e.g., electrodes #3 and 4) will sense electrophysiological signals indicative of endocardium E and the remaining proximal electrodes (e.g., electrodes #5, 6 and 7) will sense electrophysiological signals of myocardium M.

In applications where a substance is to be delivered through catheter 10, examples of the types of substances that may be so delivered include but are not limited to: contrast agents or other agents that provide an enhanced image of the target site, traceable substances that may be used to determine the rate at which the substance distributes away from or is otherwise inactivated at the target site or other pharmacokinetic or biodistributive parameters or variables, drugs, proteins, cells (e.g., stem cells, nerve cells, progenator cells, myoblasts, myocytes, secretory cells, pancreatic islet cells, dopamine secreting cells, endothelial cells, hepatocytes, cloned cells, cells grown in cell culture, genetically modified cells, and combinations thereof), angiogenic substances (e.g., vascular endothelial growth factor (VEGF), fibroblast growth factors (FGF), epidermal growth factor (EGF), platelet-derived growth factor (PDGF), hepatocyte growth factor (HGF) or scatter factor, heparin combined with an adenosine receptor agonist, nerve cell growth factor (NGF), and combinations thereof), other agents that increase vascularity of an ischemic target site, myogenic substances, neurogenic substances, genes, gene therapy compositions, genetic material in combination vectors (e.g., viruses), stem cells of a type that will mature in situ into a type of cell that is currently deficient, substances that promote the growth of myocytes in tissue that is necrotic or characterized by a lack of living myocytes, secretory cells that secrete a substance (e.g., dopamine, insulin, a particular neurotransmitter) that is deficient, step F comprises insulin secreting cells, glial cell line-derived neurotropic factor (GDNF), nerve growth factor, neuro-immunophilin ligand, poly ADP-Ribose polymerase, and combinations thereof.

In applications where an article is to be delivered through catheter 10, examples of the types of articles that may be so delivered include but are not limited to; substance eluting implants, radioactive implants, embolic members, markers, radiopaque markers, etc.

In applications where a device is to be delivered through catheter 10, examples of the types of articles that may be so delivered include but are not limited to; catheters, cannulae, guidewires, wires, electrodes, sensors, microreservoirs, implantable devices, substance eluting or delivering devices, etc.

It is to be appreciated that the invention has been described hereabove with reference to certain examples or embodiments of the invention but that various additions, deletions, alterations and modifications may be made to those examples and embodiments without departing from the intended spirit and scope of the invention. For example, any element or attribute of one embodiment or example may be incorporated into or used with another embodiment or example, unless to do so would render the embodiment or example unsuitable for its intended use. Also, where the steps of a method or process are described, listed or claimed in a particular order, such steps may be performed in any other order unless to do so would render the embodiment or example not novel, obvious to a person of ordinary skill in the relevant art or unsuitable for its intended use. All reasonable additions, deletions, modifications and alterations are to be considered equivalents of the described examples and embodiments and are to be included within the scope of the following claims.

What is claimed is:

1. A linear electrode array equipped catheter device comprising:
an elongate catheter body having a lumen and a distal end that penetrates through tissue;
a linear electrode array on or in the catheter body, said electrode array comprising a plurality of electrodes arranged in a row at spaced apart locations, each of said electrodes being operative to sense a property of tissue or body fluid and to generate signals in response to the sensed property; and
a display apparatus for displaying indicia of the sensed property.

2. A catheter device according to claim 1 wherein the linear electrode array comprises a plurality of electrodes arranged in a substantially straight row.

3. A catheter device according to claim 1 wherein the property sensed by the electrodes comprises electrophysiological signals.

4. A catheter device according to claim 1 wherein the electrode array is useable to locate an area of ischemic or infarcted tissue.

5. A catheter device according to claim 1 wherein the catheter body is substantially flexible.

6. A catheter device according to claim 1 wherein the catheter body has a lumen which terminates distally in a distal end opening through which a substance, article or device may be delivered.

7. A catheter device according to claim 1 wherein a tissue penetrating distal tip member is mounted on the distal end of the catheter body.

8. A catheter device according to claim 1 wherein the signal transmission apparatus comprises one or more wires for wired communication between the electrode array and the property display device.

9. A catheter device according to claim 1 wherein the signal transmission apparatus comprises apparatus for wireless communication between the electrode array and the property display device.

10. A catheter device according to claim 1 wherein the catheter body comprises a microcatheter body having an outer diameter of less than 24/1000 inch.

11. A system comprising a linear electrode array equipped catheter device in combination with a tissue penetrating catheter device that is positionable within a body lumen and has a tissue penetrating member that is advanceable to a first location outside of that body lumen, said linear electrode array equipped catheter device being thereafter advanceable through or over the tissue penetrating member such that the linear electrode array equipped catheter device will advance into tissue or body fluid and the electrodes of the linear electrode array will sense a property of that tissue or body fluid.

12. A system according to claim 11 wherein the tissue penetrating member of the tissue penetrating catheter comprises a needle having a hollow lumen and wherein the linear electrode array equipped catheter device is advanceable though the lumen of the tissue penetrating member.

13. A system according to claim 12 wherein the tissue penetrating catheter device comprises apparatus that indicates its rotational orientation within the body lumen relative to a target location outside of that body lumen such that the operator may adjust the rotational orientation and position of the tissue penetrating catheter device within the body lumen as needed to substantially ensure that subsequent advancement of the tissue penetrating member will cause the tissue penetrating member to travel to the desired first location and further subsequent advancement of the linear electrode array equipped catheter device through the tissue penetrating member will cause the linear electrode array equipped catheter device to enter the target location rather than some other location.

14. A system according to claim 12 wherein the tissue penetrating catheter device comprises apparatus that indicates the path on which the tissue penetrating member will subsequently advance such that the operator may adjust the rotational orientation and position of the tissue penetrating catheter device within the body lumen as needed to substantially ensure that subsequent advancement of the tissue penetrating member will cause the tissue penetrating member to travel to the desired first location and further subsequent advancement of the linear electrode array equipped catheter device through the tissue penetrating member will cause the linear electrode array equipped catheter device to enter the target location rather than some other location.

15. A method for delivering a substance, article or device to a target location within the body of a human or animal subject, said method comprising the steps of:
(A) providing a linear electrode array equipped catheter device that comprises i) an elongate catheter body having a distal end and a lumen, ii) a linear electrode array on or in the catheter body, said electrode array comprising a plurality of electrodes arranged in a row at spaced apart locations, each of said electrodes being operative to sense a property of tissue or body fluid and to generate signals in response to the sensed property and iii) a display apparatus for displaying indicia of the sensed property;
(B) inserting the linear electrode array equipped catheter device into the subject's body and advancing the linear electrode array equipped catheter device through tissue or body fluid such that the electrodes of the linear electrode array will sense a property to the tissue or body fluid and the display apparatus will display indicia of the property sensed by each electrode;
(C) determining on the basis of the indicia displayed by the display device when the catheter body is positioned such that introduction of the substance, article or device through the lumen of the catheter body will result in delivery of the substance, article or device to the target location; and
(D) delivering the substance, article or device through the lumen of the catheter body to the target location.

16. A method according to claim 15 wherein the linear electrode array equipped catheter device provided in Step A has a tissue penetrating distal tip.

17. A method according to claim 15 wherein the tissue penetrating distal tip of the linear electrode array equipped catheter device has an opening through which the substance, apparatus or device may pass and wherein;
Step C comprises determining on the basis of the indicia displayed by the display device when the distal tip of the linear electrode array equipped catheter device is positioned within or adjacent to the target location; and
Step D comprises causing the substance, article or device to pass out of the opening in the distal tip such that it is delivered to the target location.

18. A method according to claim 15 wherein Step B comprises:
providing a tissue penetrating catheter that has an advanceable and retractable tissue penetrator;
positioning the tissue penetrating catheter within a body lumen of the subject's body;
advancing the tissue penetrator from the tissue penetrating catheter to a first location outside of the body lumen in which the tissue penetrating catheter is located; and
thereafter advancing the linear electrode array equipped catheter device through or over the tissue penetrator.

19. A method according to claim 15 wherein the tissue penetrating catheter further comprises apparatus that provides an indication of the rotational orientation of the tissue penetrating catheter within the body lumen relative to the target location and wherein:
Step B further comprises using said indication of the rotational orientation to adjust at least the rotational orientation of the tissue penetrating catheter within the body lumen as needed to substantially ensure that subsequent advancement of the tissue penetrating member will cause the tissue penetrating member to travel to the desired first location rather than some other location.

20. A method according to claim 15 wherein the tissue penetrating catheter further comprises apparatus that provides an indication of the path on which the tissue penetrating member will subsequently advance and wherein:
Step B further comprises using said indication of the path on which the tissue penetrating member will subsequently advance to adjust the rotational orientation or position of the tissue penetrating catheter within the body lumen as needed to substantially ensure that subsequent advancement of the tissue penetrating member will cause the tissue penetrating member to travel to the desired first location rather than some other location.

21. A method according to claim 15 wherein the electrodes of the linear electrode array are operative to sense electrophysiological signals and wherein:
Step C comprises determining on the basis of electrophysiological signals sensed by each electrode when the catheter body of the linear electrode array equipped catheter device is positioned such that introduction of the substance, article or device through the lumen of the catheter body will result in delivery of the substance, article or device to the target location.

22. A method according to claim 15 wherein the target location comprises and area or ischemic or infarcted tissue.

23. A method according to claim 15 wherein the target location is within the myocardium.

24. A method according to claim 15 wherein the target location is within an organ.

25. A method according to claim 15 wherein the target location is within a pathological lesion.

26. A method according to claim 24 wherein the pathological lesion comprises a tumor.

27. A method according to claim 15 wherein Step D comprises delivering a substance selected from the group consisting of: drugs, proteins, cells, angiogenic substances, myogenic substances, neurogenic substances, genes, gene therapy compositions and genetic materials in combination with vectors for delivering the genetic material into locations within cells.

28. A method according to claim 27 wherein the method is carried out to improve perfusion of ischemic tissue and where the substance delivered in Step D comprises is an angiogenic agent that increases vascularity of the ischemic tissue.

29. A method according to claim 28 wherein the angiogenic agent delivered in Step D is selected from the group of angiogenic agents consisting of vascular endothelial growth factor (VEGF), fibroblast growth factors (FGF), epidermal growth factor (EGF), platelet-derived growth factor (PDGF), hepatocyte growth factor (HGF) or scatter factor, heparin combined with an adenosine receptor agonist, nerve cell growth factor (NGF), and combinations thereof.

30. A method according to claim 27 wherein the substance delivered in Step D comprises cells selected from the group consisting of stem cells, progenator cells, myoblasts, myocytes, secretory cells, pancreatic islet cells, dopamine secreting cells, endothelial cells, hepatocytes, cloned cells, cells grown in cell culture, genetically modified cells, and combinations thereof.

31. A method according to claim 30 wherein the method is carried out to treat a condition characterized by a deficiency of a type of cell that normally matures in situ from stem cells, and wherein Step D comprises delivering stem cells of a type that will mature into the deficient cell type.

32. A method according to claim 30 wherein the method is carried out to treat a condition characterized by a lack of living myocytes and wherein Step D comprises delivering myoblasts or myocytes.

33. A method according to claim 30 wherein the method is carried out to treat parkansonism and wherein Step D comprises delivering dopamine secreting cells.

34. A method according to claim 33 wherein the dopamine secreting cells comprise fetal dopamine secreting cells.

35. A method according to claim 30 wherein the method is carried out to treat diabetes and wherein Step D comprises delivering insulin secreting cells.

36. A method according to claim 35 wherein the insulin secreting cells comprise pancreatic β islet cells.

37. A method according to claim 27 wherein the method is carried out to treat a neurogenerative or nerve disorder and wherein Step D comprises delivering nerve cells.

38. A method according to claim 27 wherein the method is carried out to treat a neurogenerative or nerve disorder and wherein Step D comprises delivering a substance that facilitates nerve growth.

39. A method according to claim 38 wherein the substance delivered in Step D is selected from the group consisting of: glial cell line-derived neurotropic factor (GDNF), nerve growth factor, neuro-immunophilin ligand, poly ADP-Ribose polymerase, and combinations thereof.

40. A method according to claim 27 wherein Step D comprises delivering a gene in combination with a vector for facilitating entry of the gene into locations within cells in which the gene will have a desired therapeutic effect.

41. A method according to claim 40 wherein the vector is a virus.
